(19) United States
(12) Patent Application Publication
(10) Pub. No.: US 2004/0071791 A1
(43) Pub. Date: Apr. 15, 2004

(54) Psoriasis treatment

(76) Inventor: Jing Hua Tang, Huhhot (CN)

Correspondence Address: WILDMAN, HARROLD, ALLEN & DIXON, 225 WEST WACKER DRIVE, CHICAGO, IL, 60606, US

(21) Appl. No.: 10/268,650

(22) Filed: Oct. 10, 2002

(51) Int. Cl. A61K 35/78

(52) U.S. Cl. 424/725

(57) ABSTRACT

A composition comprising Gan Jiang, Dan Shen, Jiang Xiang, Huang Qi, Gui Zhi and Cang Zhu, is highly effective in treating psoriasis.

### FIELD OF THE INVENTION

The present invention relates to a psoriasis treatment and in particular to a traditional Chinese medicine (TCM) preparation that has been found to be highly effective in treating psoriasis and provide a greatly reduced risk of relapse.

### BACKGROUND TO THE INVENTION

Traditional Chinese Medicine (TCM) is the use of traditional Chinese medical theories and therapies, including acupuncture, herbal medicines, nutrition, massage and movement therapies (such as Tai Chi) to reinforce and stimulate the body's internal strength to cure disease.

Psoriasis is a non-contagious chronic skin disease that appears in different forms and levels of severity. It is generally characterised by patches of raised red skin covered by a flaky white buildup. Psoriasis is usually a mild condition and a number of treatments are effective in managing the clinical symptoms. Approximately seven million people in the United States suffer from psoriasis with estimated new cases numbering at about 150,000 to 260,000 new cases every year. The average age of diagnosis is 28 while psoriasis most commonly appears between 15 and 35 years of age. Cases in infants and the elderly are not uncommon. Psoriasis is generally diagnosed by examination of the skin.

The exact cause of psoriasis is unknown but it is believed to be due to faulty immune system signalling. Errant signals accelerate the production of skin cells which accumulate on the skin surface because the body is not able to shed them fast enough. Some patients have a genetic predisposition to psoriasis but an external environmental trigger is generally necessary to initiate symptoms. Identified triggers include emotional stress, injury to the skin, infections and drug reactions.

There are several forms of psoriasis including:

Plaque psoriasis;

Guttate psoriasis: small dot like lesions;

Inverse psoriasis: intense inflammation and little scaling;

Erythrodermic psoriasis: intense sloughing and inflammation of the skin;

Generalised pustular psoriasis: weeping lesions and intense scaling; and

Localised pustular psoriasis.

The most common form is plaque psoriasis with 4 out of 5 sufferers having this type. “Plaques” are well-defined patches of red, raised skin with a flaky, silvery white buildup on top called scale composed of dead skin cells. Between 10% and 30% of people with any of these forms of psoriasis also develop psoriatic arthritis. Psoriasis most commonly appears on the scalp, knees, elbows and torso but can develop anywhere including nails, palms, soles, genitals and face.

Sufferers with mild psoriasis (about 75% to 80% of sufferers) have patches of psoriasis on less than 2% of their body. Moderate to severe sufferers (about 20% to 25%) have psoriasis on upwards of 2% and 10% of their bodies respectively.

About 400 people die a year in the United States from psoriasis complications, usually due to extensive shedding of skin effecting their ability to regulate body temperature, ward off infections and retain fluids.

There is at present no recognised cure for psoriasis but many treatments exist that can ease or clear symptoms for prolonged periods. Treatments include topical therapy using steroids, calcipotrine, vitamin A, anthralin, salicylic acid, sunlight (or other photo therapy), moisturiser and bathing. Systemic medication using methotrexate, oral retinoids and cyclosporine have also been shown to be effective. However, prolonged use is either not possible or carries associated risks. Combination or rotational therapy combining the above treatments is used in serious or persistent cases.

Problems with existing remedies are that they may not be effective for all sufferers. Individuals often must receive customised treatments because their bodies react differently to different treatments. Furthermore, once treatment ceases, the risk of relapse is often high, especially on re-exposure to environmental triggers.

While TCM is known to be useful in treating psoriasis, existing TCM treatments may suffer from the same problems as treatments offered by Western medicine in that they are not effective in all cases or are not able to prevent relapse.

The present application discloses a TCM formulation found to be highly effective in treating psoriasis with a greatly reduced risk of relapse.

The present application also discloses that the treatment of the invention is more effective in patients that have never taken immune system suppressing drugs or medication which affects the production of bone marrow and white blood cells.

### OBJECT OF THE INVENTION

It is therefore an object of the invention to provide an effective TCM therapy for psoriasis; and/or to overcome or address at least one of the disadvantages of the prior art; and/or to at least provide the public with a useful choice.

### SUMMARY OF THE INVENTION

According to the broadest aspect of their invention there is provided a composition comprising Gan Jiang, Dan Shen, Jiang Xiang, Huang Qi, Gui Zhi; and Cang Zhu wherein the composition is highly effective in treating psoriasis. Preferably, the compositions also provides a greatly reduced risk of relapse.

As used herein, “highly effective” means:

1. At least 90% of patients who have never taken immune system suppressing drugs or medication which affects production of bone marrow and white blood cells recover, preferably fully; and

2. At least 80% recovery rate of patients who have taken these drugs recover, preferably fully.

As used herein, “greatly reduced risk of relapse” means skin problem areas are cured, rashes disappear, skin colour returns to normal and there is no relapse of disease for at least two years. Most patients, irrespective of whether they have taken immune system suppressing drugs or medication which affects the production of bone marrow and white blood cells, experience this result.

Preferably the composition of the invention further comprises one or more of Bai Shao, Dang Shen, and Yi Yi Ren.

According to a second aspect of the invention there is provided a method of treating psoriasis comprising administration of a composition of the invention.

Preferably the method of the invention is carried out in the absence of stimulants, anti-tumour drugs, alcohol and drugs that suppress the immune system.

According to a third aspect of the invention there is provided a use of a composition of the invention in the manufacture of a formulation for treating psoriasis.

In a preferred form the composition of the invention is prepared by mixing together the component herbs. Alternatively extracts, preferably organic extracts, of each of the components may be used.

In a preferred form of the invention, the composition of the invention further comprises one or more materials selected from the group comprising adjuvents, plasticisers, excipients, diluents, carriers, binders, lubricants, glidants and tabletting compounds. In alternative forms the composition of the invention may be included in food additives, drink additives, dietary supplements and pharmaceutical formulations. Preferably the composition of the invention is prepared in capsule form for oral administration.

The compounds useful herein are also known as:

| Chinese Name | Latin | Botanical | English |
|---|---|---|---|
| Gan Jiang | Rhizoma Zingiberis | Zingiber officinale | Dried Ginger |
| Dan Shen | Radix Salviae | Salvia miltiorrhiza | Danshen Root/Red Sage |
| | Miltiorrhizae | Bge | Root/Salvia Root |
| Jiang Xiang | Lignum Dalbergiae | Dalbergia odorifera | Rosewood Heart Wood |
| | Odoriferae | T. Chen | |
| Huang Qi | Radix Astragali | Astragalus | Astragalus Root/Membranous |
| | | membranaceus | Milkvetch Root/Mongolian |
| | | (Fisch.) Bge. | Milkvetch Root |
| Gui Zhi | Ramulus | Cinnamomum cassia | Cassia Twig, |
| | Cinnamomi | Presl | Cinnamon Twig |
| Cang Zhu | Rhizoma | Atractylodes lancea | Swordlike Atractylodes |
| | Atractylodis | Rhizome | Rhizome/Chinese |
| | Lanceae | | Atractylodes Rhizome |
| Bai Shao | Radix Paeoniae | Paeonia lactiflora | White Paeony Root |
| | Alba | Pall | |
| Dang Shen | Radix Codonopsis | Codonopsis pilosula | Pilose Asiabell Root/ |
| | Pilosulae | Nannf. | Moderate Asiabell Root/ |
| | | | Szechwon Tangshen Root |
| Yi Yi Ren | Semen Coicis | Coix lacrymajobi L. | Coix Seed |
| | | ma-yuen Staph. | |

This invention may also be said broadly to consist in the parts, elements and features referred to or indicated in the specification of the application, individually or collectively, and any or all combinations of any two or more of said parts, elements or said features, and where specific integers are mentioned herein which have no equivalents in the art to which this invention relates, such known equivalents are deemed to be incorporated herein as if individually set forth.

The invention consists of the forgoing and also envisages constructions of which the following gives examples.

### DETAILED DESCRIPTION OF THE INVENTION

Psoriasis a chronic disease of the skin with a high tendency of relapse. Reports have estimated that there are presently approximately 50 million patients worldwide. There is a need for an effective treatment of psoriasis that minimises relapse.

Presently, clinical treatment for psoriasis is usually carried out using stimulants or tumour-control drugs. However, these drugs only provide temporary relief and do not cure the patient of the disease. The majority of these drugs affect the production of bone marrow and white blood cells. Patients are also required to abstain from many types of food. Medication of this type may lead to complications and hinders the normal functioning of the immune system.

After more than 20 years of clinical observation, it is observed that besides itchiness, skin damage and other skin problems, most psoriasis patients will also prefer warmth to cold. This phenomenon is especially obvious in patients whos conditions are more serious. The patients either perspire very little or not at all. According to the theory of Traditional Chinese Medicine (TCM), weakness in the “Yang” will lead to sensitivity to external cold while weakness in the “Yin” will lead to sensitivity to internal heat. The composition of the present invention is based on the therapeutic method of warming the “Yang” and promoting blood circulation. The composition of the invention has found to be highly effective in treating psoriasis with a greatly reduced risk of relapse.

No Previous Use of Immune System Suppressing Drug

Patients who have never been treated with immunosuppressants will benefit the most from the composition of the present invention. Patients will first notice improvement of symptoms beginning from the upper part of the body and gradually moving downwards upon taking the composition of the present invention. About a week after first treatment, new rashes will stop forming and old rashes (thick and scaly rough patches) will improve. With continuing treatment, the thick and scaly skin patches will reduce and thin down. Upon recovery, the affected skin becomes fair and will take on the tone of healthy skin. Patients will usually achieve a full recovery within three months with no relapse. Patients in this category show approximately a 96% success rate with the treatment using the composition of the invention.

Previous Treatment with (Immune System Suppressing Drug)

For patients who have previously been treated with immunosuppressants, improvements of their symptoms will begin from the lower part of the body gradually moving upwards upon taking the composition of the invention. A small number of patients may see improvement in all parts of the body concurrently. About 30 days after beginning treatment, some new onset rashes will be surrounded by a white circle while rashes will cease gradually and the thick and scaly skin patches will reduce and thin down. Patients in this category show approximately an 87% success rate using the composition of the invention. The patient's affected areas will take on the tone of healthy skin last.

After the old rashes in the form of thick and scaly skin patches, the affected area becomes greyish and in some cases it will take more than two years before these areas take on the tone of healthy skin. It is believed this is due to the adverse affect of immunosuppressants.

For patients where the affected areas do not become totally fair (healthy skin toned), it has been observed that relapse may occur. Where old rashes disappear and the affected area turns fair this indicates full recovery and no relapse will occur.

Approximately 6% to 7% of patients exhibit a ring recovery style where the rashes thin down and disappear towards a centre point. Patients of this category will take a relatively longer time to reach a full recovery and the patient's head area will take on the tone of healthy skin lastly.

The following example reports results observed from use of the composition of the invention by 252 patients.

### EXAMPLE

Clinical Information

Patient's presented the following clinical information:

(a) The patients' skin disorder exhibited in the form of patches of raised, rough, red and scaly rashes. On the surface were multi-layered silvery white scaly rashes and these were accompanied by mild or serious itchiness.

(b) This condition often spread to the whole body, especially the hands, legs and knees, in the form of small uneven patches.

(c) Acute breakout with a tendency of relapse was common and usually related to seasonal changes.

(d) Heredity played a part in some patients.

Treatment Group

The treatment group comprised 136 males and 116 females, 252 patients in total. The following tables provide further information on the characteristics of the treatment group.

| | Number of cases |
|---|---|
| Age | |
| 1-10 | 24 |
| 11-20 | 71 |
| 21-30 | 96 |
| 31-40 | 41 |
| 41-50 | 12 |
| 50+ | 8 |
| TOTAL | 252 |
| Illness duration | |
| 0-1 year (shortest) | 50 |
| 2-5 years (most common) | 90 |
| 6-15 years (second most common) | 73 |
| 31-38 years (longest) | 2 |
| TOTAL | 215 |
| Age upon treatment | |
| 6-16 years | 29 |
| 17-36 years | 145 |
| 37-56 years | 75 |
| 57+ years | 13 |
| TOTAL | 262 |

The 252 patients were able to be divided into two groups:

Group 1: Patients who have never taken immune suppressing drugs or medication that controls the production of bone marrow and white blood cells.

63 patients

Group 2: Patients who have taken this medication prior to being treated with a composition of the present invention.

189 patients

Treatment Method

A composition comprising Gan Jiang, Dan Shen, Jiang Xiang, Huang Qi, Gui Zhi and Cang Zhu was used.

The composition was administered in capsule form to both Groups 1 and 2. An equal dosage of the capsule was administered at the same time to each patient, three times a day. Each phase consisted of a 30 day treatment with five capsules per dosage. Patients may require up to three phases of treatment, depending on their individual requirements.

Reaction to Treatment

The following results were observed:

Result A: Skin problems were completely cured, rashes disappeared, skin colour returned to normal, and there was no relapse of the disease for at least two years.

Result B: Skin problem areas were cured but skin colour did not return to normal and disease relapse occurred several months later.

| Group | Result A | | Result B | | Total |
|---|---|---|---|---|---|
| 1 | 60 | cases (95.2%) | 3 | cases (4.8%) | 63 |
| 2 | 164 | cases (86.8%) | 25 | cases (13.2%) | 189 |
| TOTAL | 224 | (88.9%) | 28 | (11.1%) | 252 |

Comparison of the results between the two groups gave P<0.05.

Analysis

About 95% of patients in Group 1 were cured completely after about three months of treatment, regardless of the length of time they had suffered from psoriasis and their age when they first contracted the disease. During the treatment process, recovery began from the head downwards without any relapse. The areas with skin problems turned white gradually and returned to normal skin colour after about a month. After medication was withdrawn there was no relapse for at least 2 years.

Patients in Group 2 took a much longer period to achieve recovery compared to Group 1 patients. Recovery took place throughout the whole body concurrently for some patients, while for others, recovery occurred gradually from the legs upwards. There was frequent relapse during the treatment process but the conditions generally improved after each relapse. When the condition improved, the problem skin areas first turned a greyish brown and often took at least several months to return to normal skin colour. Continued treatment with the composition of the invention was required until the skin fully recovered. Approximately 87% of patients in Group 2 recovered.

The above describes some preferred embodiments of the invention and indicates several possible modifications but it will be appreciated by those skilled in the art that other modifications can be made without departing from the scope of the invention.

What we claim is:

1. A composition comprising Gan Jiang, Dan Shen, Jiang Xiang, Huang Qi, Gui Zhi and Cang Zhu, wherein the composition is highly effective in treating psoriasis.

2. A composition of claim 1 which provides a greatly reduced risk of relapse.

3. A composition of claim 1 or 2 further comprising one or more of Bai Shao, Dang Shen and Yi Yi Ren.

4. A composition of any one of claims 1 to 3 further comprising one or more of adjuvents, plasticisers, excipients, diluents, carriers, binders, lubricants, glidants and tabletting compounds.

5. A method of treating psoriasis comprising administering a composition of any one of claims 1 to 4 to a patient in need thereof.

6. A method of claim 5 wherein said method is carried out in the absence of stimulants, anti-tumour drugs and/or drugs that suppress the immune system.

7. A use of a composition of claim 1 to 4 in the manufacture of a formulation for treating psoriasis.

8. A composition as claimed in any one of claims 1 to 4 substantially as herein described.

9. A method as claimed in claim 5 or 6 substantially as herein described.

10. A use as claimed in claim 7 substantially as herein described.
